(19) United States
(12) Patent Application Publication
(10) Pub. No.: US 2013/0116544 A1
(43) Pub. Date: May 9, 2013

(54) DUAL PRESSURE SENSOR SIGNAL CHAIN TO REMOVE MUTUALLY-COUPLED MRI INTERFERENCE

(71) Applicants: Eduardo Rey, Winter Springs, FL (US); Harold Cates, Orlando, FL (US)

(72) Inventors: Eduardo Rey, Winter Springs, FL (US); Harold Cates, Orlando, FL (US)

(73) Assignee: KONINKLIJKE PHILIPS ELECTRONICS N.V., EINDHOVEN (NL)

(21) Appl. No.: 13/810,903

(22) PCT Filed: Jul. 12, 2011
(86) PCT No.: PCT/IB2011/053093

§ 371 Date: Jan. 18, 2013

Related U.S. Application Data
(60) Provisional application No. 61/366,988, filed on Jul. 23, 2010.

(51) Int. Cl. A61B 5/0205; A61B 5/00; A61B 5/113; A61B 5/055

(52) U.S. Cl. CPC A61B 5/0205; A61B 5/055; A61B 5/725; A61B 5/7225; A61B 5/1135; A61B 5/6831; A61B 5/7285; A61B 5/0004; A61B 5/0059; A61B 5/0077
USPC 600/411

(57) ABSTRACT

Apparatus and methods provide a physiological status sensing device (40) for sensing a physiological status of a patient (4) and minimizing an amount of interference (78) generated during a resonance (MR) scan by a magnetic resonance (MR) system (8). The device (40) includes a first, active sensor (64) located to sense the physiological status and experience MR scan related interference and to generate a first signal (80) having a physiological status component (76) and an interference component (78). A second non-active sensor (70) is located closely adjacent to the first sensor (64) to experience substantially the same MR scan related interference (78) as the first sensor (64) and generate a second signal (82) having only the interference component (78). A circuit or processor (56, 84, 110, 116) subtractively combines the first (80) and second signals (82) to cancel the interference component (78).

The following relates to the medical arts, magnetic resonance arts, physiological monitoring arts, and related arts. It finds application in magnetic resonance imaging and other magnetic resonance applications that are beneficially monitored by electrocardiography, and the like.

When a patient is undergoing a magnetic resonance imaging (MRI) scan procedure, the patient is position in a static magnetic field (B₀), e.g., inside the bore of the MRI scanner. Radiofrequency fields (B₁), and gradient magnetic field pulses (Gₓ, G_{y}, G_{z}) are applied and directed at the patient to induce resonance, spatially localize resonance signals, and the like. Electro medical devices inside or near the MRI bore, such as patient monitors and life support devices, are exposed to these types of fields. The B₀ field acts as a strong electromagnet that attracts ferromagnetic objects towards the magnet. The B₀ field is typically present as long as the magnet is powered (even when scans are not being performed). In addition, the B₁ field is generated by an RF coil that emits radiofrequency waves into free space at frequencies related by the Larmor equation. These frequencies are, for example, approximately 64 MHz and 128 MHz for 1.5 T and 3.0 T magnets, respectively. The gradient fields are generated by gradient coils that momentarily alter the static magnetic field (B₀) to produce a momentary change in magnetic field strength. Unlike the static field (B₀), the radio frequency (RF) field (B₁) and the MRI gradients (Gₓ, G_{y}, G_{z}) are generated and used only during actual scan sequences. The combination of these three fields allow for image reconstruction.

MR-compatible products must operate within specifications in the presence of these interference sources. To avoid this interference, respiration monitors, for example, commonly include a bladder, which is mounted to or around a patient's waist within the fields. A pneumatic tube connects the bladder with appropriate electronics mounted away from the scanner, e.g., beyond the 5 gauss line, outside the shielded room, or the like. Other monitors use light, such as lasers, video cameras, or fiber optics, to sense physiological status and convey the monitored status to remote electronics.

The following provides a new and improved apparatus and methods which overcome the challenges discussed above and others.

In accordance with one aspect, a physiological status sensing device senses a physiological status of a patient and minimizes an amount of interference generated during a magnetic resonance scan. The device includes a first, active sensor which is located to sense the physiological status of the patient, experience the MR scan related interference, and output a first signal having a physiological status component and an interference component. A second non-active sensor is located closely adjacent to the first sensor to experience substantially the same MR related interference as the first sensor and output a second signal having the interference component. A circuit subtractively operates on the first and second signals to cancel the interference component.

In accordance with another aspect, a method for sensing the physiological status of a patient and minimizing an amount of interference generated during a magnetic resonance scan is provided. The physiological status and magnetic scan related interference are sensed by a first, active sensor to generate a first signal having a physiological status component and an interference component. The magnetic resonance scan related interference is also sensed by a second, non-active sensor positioned closely adjacent the first sensor to experience substantially the same MR scan related interference as the first sensor to generate a second signal having the interference component. The first and second signals are subtractively combined to cancel the interference component and generate a signal with the physiological status component.

One advantage resides in enabling sensor electronics to be placed in high field regions.

Another advantage resides in artifact-free or reduced physiological sensor signals.

Another advantage resides in compact size and elimination of pneumatic cabling.

Further advantages of the present invention will be appreciated to those of ordinary skill in the art upon reading and understand the following detailed description.

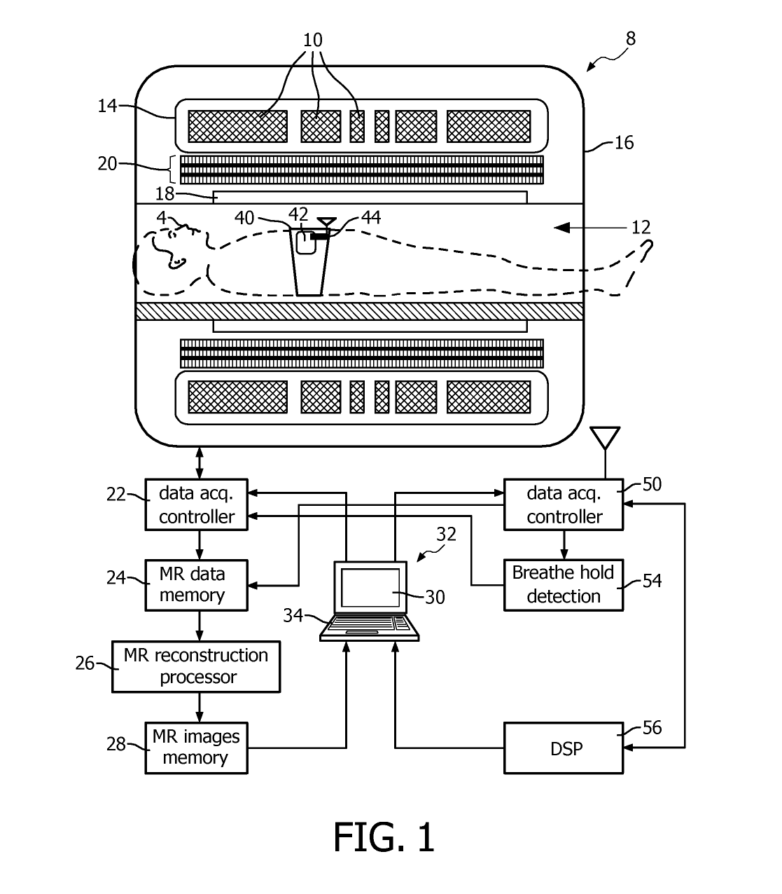

FIG. 1 diagrammatically illustrates a magnetic resonance data acquisition system.

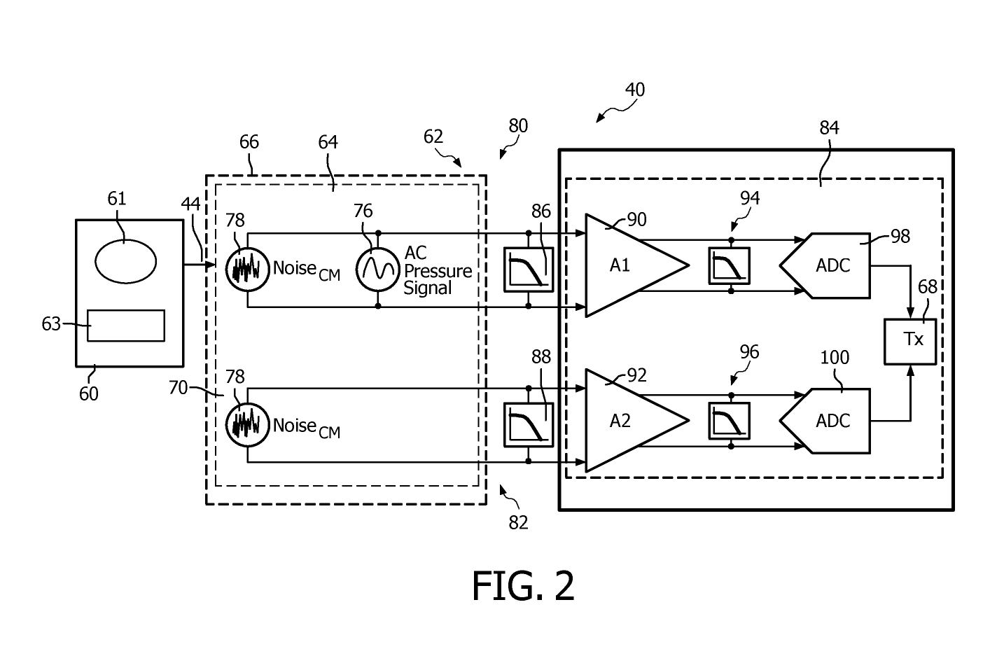

FIG. 2 diagrammatically illustrates one embodiment of a respiratory sensor for use in the system of FIG. 1 in combination with an interference filter for removing interference.

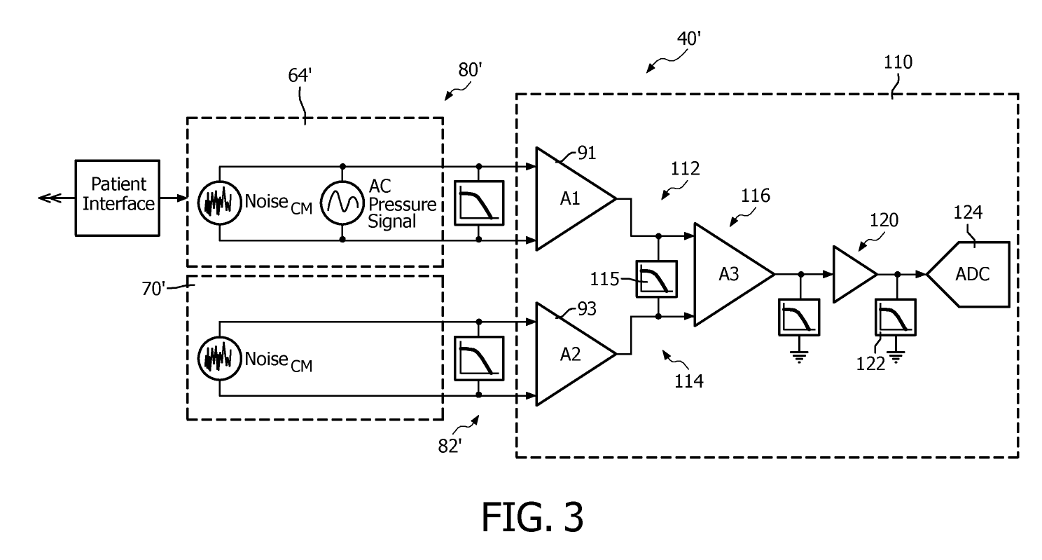

FIG. 3 diagrammatically illustrates one embodiment of a sensor for use in the system of FIG. 1 in combination with an interference filter for removing interference.

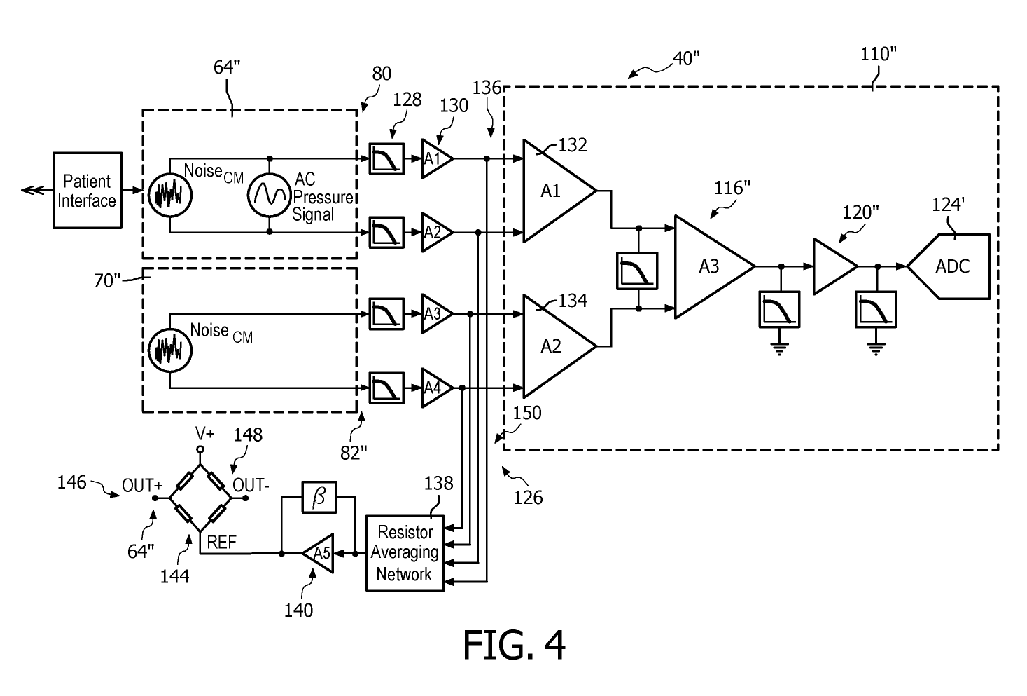

FIG. 4 diagrammatically illustrates one embodiment of a sensor for use in the system of FIG. 1 in combination with an interference filter for removing interference.

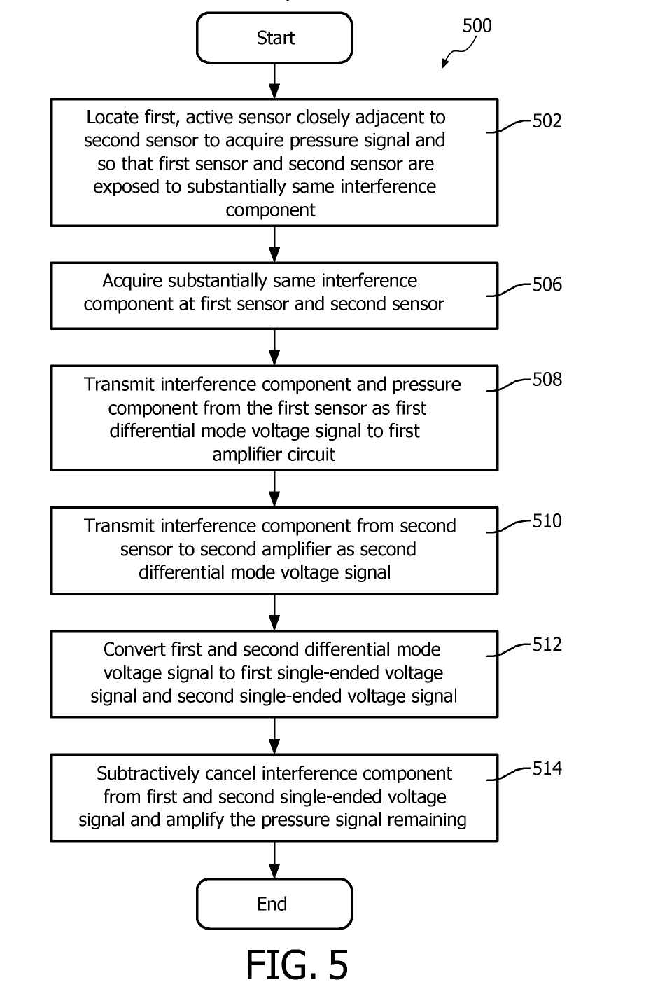

FIG. 5 illustrates a method of monitoring physiological status of a patient in an MR environment.

Gradient fields (Gₓ, G_{y}, G_{z}), in particular, are a primary source of MRI interference in other electro-medical devices operating inside or near an MRI bore. In a three-dimensional space, when an MR imaging gradient is introduced, this appears as a fast or pulsed change in field strength that is superimposed across the patient to allow spatial localization. During this time, MRI gradient interference is inductively coupled into electro-medical devices (via loop or dipole-antenna effects) and can appear as differential-mode (DM) or common-mode (CM) interference. Therefore, the MRI gradients generated by the gradient field magnet are a primary interference source and the electronics on the Printed Circuit Assembly (PCA) are the victim. Because the MRI system uses a highly homogenous static field and the MRI gradients appear as wavefronts across the patient or a specific part of the patient, any two locations, near or inside the bore, exposed to the gradient field pulses that are relatively close to each other and oriented in the same direction, will be exposed to similar or highly correlated electromagnetic (EM) fields. These similar electromagnetic fields are referred to herein as “mutually-coupled electromagnetic interference.” With this presumption of nearby devices being exposed to similar gradient fields, one embodiment of the present disclosure utilizes two pressure sensors mounted adjacent to each other and coupled to a signal chain architecture, to remove the electromagnetic interference that is mutually-coupled to both sensors.

With reference to FIG. 1, a magnetic resonance (MR) system includes a MR scanner 8 having a main magnet 10 that generates a static main (B₀) magnetic field in an examination region 12. In the illustrated embodiment, the main magnet 10 is a superconducting magnet disposed in a cryogenic vessel 14 employing helium or another cyrogenic fluid; alternatively a resistive main magnet can be used. In the illustrated embodiment, the magnet assembly 10, 14 is disposed in a generally cylindrical scanner housing 16 defining the examination region as a bore 12, such as a cylindrical bore; alternatively, other geometries such as an open or other MR geometry can also be used. Magnetic resonance is excited and detected by one or more radio frequency coils (B₁), such as an illustrated whole-body quadrature body coil 18 or one or more local coils or coil arrays such as a head coil or chest coil. The excited magnetic resonance is spatially encoded, phase- and/or frequency-shifted, or otherwise manipulated by magnetic field gradients selectively generated by a set of magnetic field gradient coils 20.

The magnetic resonance scanner 8 is operated by a magnetic resonance data acquisition controller 22, suitably embodied by a dedicated digital processing device, a suitably programmed general purpose computer, or so forth, to generate, spatially encode, and read out magnetic resonance data, such as projections or k-space samples, that are stored in a magnetic resonance data memory 24. The acquired spatially encoded magnetic resonance data are reconstructed by a magnetic resonance reconstruction processor 26 to generate one or more images of a patient or subject 4 disposed in the examination region 12. The reconstruction processor 26 employs a reconstruction algorithm comporting with the spatial encoding, such as a backprojection-based algorithm for reconstructing acquired projection data, or a Fourier transform-based algorithm for reconstructing k-space samples. The one or more reconstructed images are stored in a magnetic resonance images memory 28, and are suitably displayed on a display 30 of a computer system 32, or printed using a printer or other marking engine, or transmitted via the Internet or a digital hospital network, or stored on a magnetic disk or other archival storage, or otherwise utilized. The illustrated computer system 32 also includes one or more user input devices such as an illustrated keyboard 34, or a mouse or other pointing-type input device, or so forth, which enables a radiologist, or other clinician user to manipulate images and, in the illustrated embodiment, interface with the magnetic resonance scanner controller 22.

With continuing reference to FIG. 1, the MR system includes a physiological status sensing device 40, such as a pressure sensing device that includes one or more pressure transducers 42 operatively connected to the computer system 32. The pressure transducer(s) may be be included in a respiratory sensor, an invasive or non-invasive blood pressure sensor, or any other medical device operative to utilize pressure, light, or other non-electrical medium for acquiring patient data inside the bore 12 of the MR system. In one embodiment the physiological status sensing device 40 includes a belt or cuff 44 that extends around a patient's torso or a limb. The physiological status sensing device 40 senses pressure stimuli within the bore 12 and then wirelessly sends data to a monitoring signal acquisition device 50. The physiological status sensing device 40 further includes a wireless transmitter (not shown) for sending wireless pressure signals to the monitoring signal acquisition device 50. The present disclosure is not limited to a wireless telemetry and may also include a fiber optic, body coupled near field, wired solutions, or the like.

The monitor signal acquisition device 50, in one embodiment, acquires pressure signals from the physiological status sensing device 40, determines a respiratory or other physiological state, and stores the physiological state with the MR data in the MR data memory 24. This physiological state information can be used for example in retrospective gating to sort the data by physiological state. In this manner, the MR data can be reconstructed based on respiratory or other physiological state.

In another embodiment, a breath hold detection circuit 54 detects the patient's breathe hold status and communicates the breathhold status to the MR data acquisition controller 22. The MR data acquisition controller can use the breathhold status for prospective gating to limit data acquisition to a breathhold or other respiratory state. A digital signal processor 56 is also included for processing the physiological status signals, for example, to generate a respiratory cycle display on the monitor 30. The present disclosure is not limited to respiratory monitoring, but is operable with any medical device for sensing pneumatic or other stimulus and processing the signals therefrom.

With reference to FIG. 2, illustrates an exemplary embodiment of the physiological status sensing device 40 having a patient interface 60, a signal processing chain 62 coupled thereto, and a transmitter 68. The patient interface 60 comprises an invasive or non-invasive measuring element, for example, for a blood pressure module such as a blood pressure dome 63, or a pneumatic respiratory measurement system such as a bladder 61. In the respiratory example, a belt or strap 44 extends around the patient's chest. As the patient's chest expands and contracts during breathing, the pressure in the bladder increases and decreases. A first or active pressure to electrical signal transducer 64, such as a piezoresistive sensor, converts the breathing related pressure variations into electrical signals.

The first or active sensor 64 is mounted on a sensor module 66 with a second or dummy sensor 70. The first sensor 64 and second sensor 70 are substantially identical pressure-to-electric converters, such as piezoesistive pressure transducers located closely adjacent to one another in a same direction and a same axis so that each sensor is exposed to a same amount of interferene, such as a same magnetic gradients (Gₓ, G_{y}, G_{z}) having substantially the same amplitudes and phases with one another. While both sensors are mounted adjacent to each other, only the first is active and actually connected to the bladder pressure dome to monitor pressure changes. The other second is a non-active (i.e., a dummy) sensor which is mounted so that it does not sense the pressure changes. In this manner, this first, active sensor 64 generates an output electrical signal which is the sum of a pressure component 76 indicative of pressure changes and a noise signal 78 indicative of the magnetic gradient interference, which is generated during actual scanning processes. The second, inactive sensor 70 outputs the same noise signal 78, but has no pressure component.

In one embodiment, the first sensor 64 and the second sensor 70 comprise piezoresistive Wheatstone bridge circuits, which can be utilized for invasive pressure monitoring applications, for example. Wheatstone bridge circuits have a differential-mode voltage output that is proportional to a change in pressure. In this embodiment, the first sensor 64 and the second sensor 70 generate a first differential output 80 and a second differential output 82, respectively, with each having a differential-mode output voltage. Typically, Wheatstone bridge circuits are constructed from four resistors, one of which has a calibration value (Rₓ), one of which is variable (R₂), e.g. with changes in pressure, and two of which are fixed and equal (R₁ and R₃), connected as the sides of a square. Two opposite corners of the square are connected to a source of electrical current, such as a battery. The other two opposite corners output the differential output. Wheatstone bridge circuits are well known by one of ordinary skill in the art and provide many applications with principles that are not therefore explained in great detail herein.

The first differential output 80 and second differential output 82 from the first and second sensors, respectively, are coupled to an interference filter 84 with a first differential mode and common mode low pass filter 86 and a second differential mode and common mode low pass filter 88, respectively, coupled therebetween. The filters 86 and 88 comprise differential mode filters that filter any signal that is differential in nature, such as the AC signal 76 generated by the pressure stimuli and any noise 78 that is electromagnetically coupled to both sensors. Both of the signals 76 and 78 are differential in nature (i.e., transmitted with two complementary signals), with a spectral band width of zero to 10 hertz for the pressure stimulus signal 76 and the coupled noise signal 78 extending up to about 30 kilohertz, for example. Consequentially, the differential mode filter serves to filter both of these signals. The electromagnetic interference components above a cutoff frequency above the frequency of the pressure signals, e.g. about 1 kilohertz, for example, are removed. This leaves the part of the noise signal that is in the same bandwidth as the pressure signal. The common mode part of the first and second filters addresses noise from pathways and devices of both signal paths (e.g., the sensor devices and differential output connections) in addition to any external interference.

With continued reference to FIG. 2, the interference filter 84 includes a first instrument amplifier 90 and a second interference amplifier 92 respectively coupled to the first differential output 80 and the second differential output 82. The first and second instrument amplifiers 90, 92 each include a differential amplifier, which is a type of electronic device that multiplies the difference between two inputs by a constant factor or differential gain. A third and fourth differential mode and common mode low pass filter 94, 96 further filters the differential outputs of respective amplifiers 90 and 92 before analog signals including the noise and AC pressure signal are convertered at converters 98 and 100. The converters 98 and 100 include fully differential analog to digital converters for converting analog signals to digital signals. These digital signals are afterwards either transmitted by the radio frequency transmitter 68 wirelessly at a frequency that does interfere with the MR system scanning. Alternatively, the digital signals are sent out on an optic fiber, or other communication link (not shown). The DSP 56 discussed in conjunction with FIG. 1, in one embodiment, filters the digital signal to remove the remaining noise components of the signal by appropriate algorithms so that the pressure signal is locally and/or remotely displayed, for example, at display 30. For example, the signal from converter 98 has a pressure signal component and a noise component and the signal from converter 100 has only the noise component. Because both noise components are the same, subtractively combining the signals leaves the pressure signal component. Consequently, two differential digital signals produced as output to the converters 98 and 100 are transmitted out of the bore and to a remote signal processing area. The DSP 56 could be located inside or outside the bore 12 of the MR system. MR imaging gradient artifacts and other interfering signals are thus effectively rejected and the stimulus pressure waveform is captured as a strong, clean signal.

The wiring of the pressure sensing device preferable has a length less than 5 cm to minimize a potential for the B₁ fields including current therein.

With reference to FIG. 3, another embodiment of a pressure sensing device 40′ for minimizing an amount of interference generated during an MR scan in an MR system is illustrated. The device 40′ includes a signal chain architecture operable as an interference filter 110 for filtering mutually-coupled MR imaging (MRI) interference. A first differential output 80′ and a second differential output 82′ from a pressure sensing transducer 64′ and a like dummy transducer 70′ shielded from sensing pressure are supplied to the interference filter 110.

The interference filter 110 includes a first instrument amplifier 91 and a second instrument amplifier 93 having a third output 112 and a fourth output 114, respectively. Each amplifier includes a differential amplifier that receives a differential-mode output voltage and is configured to convert the voltage to a single-ended voltage output at the third and fourth outputs respectively. Each single-ended voltage is therefore generated from the outputs of an active pressure transducer 64′ and a non-active pressure transducer 70′.

A third amplifier 116 receives as an input each single-ended output voltage from the first and second amplifiers 91, 93 after being filtered by a third filter 115. The third amplifier 116 subtracts the common mode signals to cancel the noise that is common to both inputs while amplifying the differential signals via the high common mode reduction capability therein, e.g., around 95 db at a unity gain or gain of zero decibels. For example, the third amplifier 116 is an instrumentation amplifier that removes the common mode noise, amplifies the differential, and then transmits a clean pressure signal downstream to an ADC driver 120 with an ADC filter 122, which decouples charge from the sampling nature of a converter 124. The converter 124 includes an analog digital converter to convert the analog signals to digital format for further processing and transmission. Low pass filters are used to remove high frequency noise generated by the system and devices therein to achieve a deeper roll off of the pressure signal.

The digital signal generated from the converter 124 can be either transmitted by an RF transmitter (not shown) that does not interfere with the MR system, an optic fiber, or other communication link for digital processing. In addition, the digital signal could be transmitted from within the bore and to a local display or external interface for viewing.

In FIG. 4, like elements with FIGS. 2 and 3 are denoted by the same reference numeral followed by a double prime (″). An active drive stage 126 integrated into a pressure sensing device 40″ for further interference rejection is illustrated. Dual pressure sensors including an active pressure sensor 68″ and a non-active pressure sensor 70″ that is prevented from sensing pressure are electromagnetically coupled to one another to transmit a similar differential noise signal at four different output terminals. A first pair of output terminals define a positive and negative differential mode voltage terminal pair 80″ which carry an AC pressure signal captured by the first sensor and a noise component. A second pair of output terminals define a differential mode pair 82″ which carry the component. A low pass filter 128 filters higher frequency signals at each terminal, which is interfaced with the active drive stage 126.

The active drive stage 126 includes high impedance buffers 130 that receive each positive and negative compliment of the differential signals before interfacing with a first amplifier 132 and a second amplifier 134. The buffers 130 are single-ended buffers that respectively receive each plus or minus differential voltage generated by the sensors 64″ and 70″. Each output of differential outputs 80″ and 82″ are processed independently and fed to the first and second amplifiers 132 and 134, respectively. The outputs of amplifiers 132, 134 are subtractively combined by amplifier 116″ and converted to a digital pressure signal by an analog-to-digital converter 124″. Consequently, the inputs to the instrument amplifiers 132 and 134 receive low impedance output signals, rather than a moderate impedance input source that they would receive if they were more directly interfaced to the pressure transducers 68″ and 70″ as illustrated in FIG. 3. The outputs of the buffers 130 are also connected with a tap 150 for directing the high impedance signal to a resistor averaging network 138.

The high impedance output signals are averaged by the resistor averaging network 138 where each signal that is common to all the outputs of the buffers 130 is averaged with the other signals and outputted to an inverter 140 having a gain β device in parallel providing a gain factor β to the signal. The signal is further provided to a reference voltage terminal 144 to the active pressure sensor 68″, which is illustrated as a Wheatstone bridge circuit with a reference voltage terminal 144 and a plus and minus differential voltage output terminals 146, 148 of the differential mode voltage output terminal 80″. The same reference signal from the inverter 140 can be fed to the reference voltage terminal of the Wheatstone bridge of the dummy pressure transducer 70″.

An example methodology 500 for removing interference from within an MRI bore during scanning is illustrated in FIG. 5. While the method 500 is illustrated and described below as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

The method 500 initializes at start and at 502 a pressure signal is acquired by locating a first, active sensor. In one embodiment, the first sensor is located closely adjacent to the second, dummy sensor so that the first sensor and second sensor are exposed to an interference that is substantially equal or the same in amplitude and phase. In one embodiment, two sensors are piezoresistive Wheatstone bridge circuits that are intended for invasive pressure monitoring applications. Physically, both pressure transducers are mounted closely adjacent to each other in the same direction and axis so that both sensors are exposed to similar levels of interference, such as the gradient pulse fields. While both sensors are mounted adjacent to each other, only one is actually receiving a pressure stimulus and the other is prevented from receiving a pressure stimulus. Both receive highly correlated electromagnetic interference. Wheatstone bridge circuits have a differential-mode voltage output that is proportional to a change in pressure. Since there are two sensors, there are two differential voltages at the input to the analog front-end. Therefore, the pressure signal together with interference is transmitted from the first sensor as a first differential mode voltage signal to a circuit having a first amplifier circuit at 508, and the second sensor transmits a second differential mode voltage signal to a second amplifier at 510.

In one embodiment, the first and second amplifiers are two high-speed instrumentation amplifiers that convert the differential-mode output voltage from the two pressure transducers into two single-ended voltages at 512; alternatively, the outputs could be differential outputs if the amplifiers are differential amplifiers. In this stage, both amplifiers capture similar interfering fields, while only one also captures a pressure signal.

At 514 the interference that is common mode interference of both the first and second differential mode voltage signals is canceled by subtracting the interference component from the first and second signals. For example, the single ended mode voltage signals are operatively connected to a difference amplifier. Because the instrumentation amplifier is a difference amplifier, the next stage, another instrumentation amplifier, for example, takes the difference between these two signals which are: [Pressure Signal+Noise_{cm}] and [Noise_{cm}]. In another embodiment, the signals are digitized and digitally subtracted.

An advantage of the method is that it is effective in cancelling MRI gradient artifact in various applications that use a piezoresistive pressure transducer and is not limited to any one particular application or field of application. The pressure signal consequently can be wireless transmitted from within the bore of an MR system. A majority of interference is inductively coupled at the pressure transducer through Faraday's law of induction and effectively canceled.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A physiological status sensing device for sensing a physiological status of a patient and minimizing an amount of interference generated during a magnetic resonance (MR) scan by a magnetic resonance (MR) system, the device comprising:
a first, active sensor located to sense the physiological status and experience MR scan related interference and to output a first signal having a physiological status component and an interference component;
a second, non-active sensor located closely adjacent to the first sensor to experience substantially the same MR scan related interference as the first sensor and output a second signal having the interference component; and
a circuit which subtractively operates on the first and second signals to cancel the interference component.

2. The device according to claim 1, wherein the first sensor includes a first piezoresistive pressure transducer which senses a pressure related to the physiological status and the second sensor includes a second piezoresistive pressure transducer of like construction with the first piezoelectric pressure transducer, the second pressure transducer being positioned to experience substantially the same MR scan related interference as the first piezoresistive transducer and not sense the pressure.

3. The device according to claim 1, wherein the first and second sensors have like construction and are mounted with a common orientation to a common substrate.

4. The device according to claim 1 further including:
an interference filter unit that is electrically coupled to the first signal having a first differential output and the second signal having a second differential output of the first sensor and the second sensor respectively, and removes the interference component from the first and the second differential output, and transmits the pressure signal, the first and the second differential output each have complementary output signals transmitted thereat.

5. The device according to claim 1 wherein the circuit includes:
a first amplifier coupled to the first sensor and having a third output;
a second amplifier coupled to the second sensor and having a fourth output;
a differential amplifier which subtractively combines the third and fourth outputs to generate a pressure signal; and
a converter which digitizes the pressure signal.

6. The device according to claim 1, wherein at least one of the active sensor and the non-active sensor are in a Wheatstone bridge and further including:
an active driver stage coupled to a reference terminal of the first, active sensor and including:
high impedance buffers coupled to the first sensor and the second non-active sensor to provide a high impedance output at each sensor output;
a resistor averaging network that averages each high impedance output of the high impedance buffers and generates an averaged signal therefrom; and
a gain device coupled to an inverter that inverts the averaged signal and biases the Wheatstone bridge with the inverted averaged signal.

7. The device according to claim 1, wherein the physiological status is a respiratory state and function including:
a belt or strap configured to encircle the patient's waist;
a bladder mounted to the belt to be compressed during the patient's respiratory cycle; and
the first sensor connected to the bladder to sense pressure therein.

8. The device according to claim 1, wherein the physiological status is a blood pressure state and further including:
a dome mounted to be compressed with changes in blood pressure; and
the first sensor connected to the dome to sense pressure therein.

9. An MRI system comprising:
a main magnet which generates a static magnetic field in a patient;
gradient coils which imposes or imposing gradient magnetic fields on the static magnetic field;
radio frequency coils which induces radio frequency fields;
a controller which controls the gradient coils and the radiofrequency coils to acquire magnetic resonance information from the patient; and
the physiological status sensing device according to claim 1.

10. The MRI system according to claim 9, wherein the controller receives the output of the physiological status sensing device and controls the gradient and radiofrequency coils to acquire magnetic resonance information during a preselected physiological status of the patient.

11. The MRI system according to claim 9, further including:
a reconstruction processor which receives the output of the physiological status sensing device and reconstructs images of the patient in one or more selected physiological status.

12. A method for sensing a physiological status of a patient and minimizing an amount of interference generated during a magnetic resonance (MR) scan, the method comprising:
with a first, active sensor sensing the physiological status and MR scan related interference and to generate a first signal having a physiological status component and an interference component;
with a second non-active sensor positioned closely adjacent to the first sensor to experience substantially the same MR scan related interference as the first sensor sensing the MR scan related interference and generating a second signal having the interference component; and
subtractively combining the first and second signals to cancel the interference component and generate a signal with the physiological status component.

13. The method according to claim 12, further including:
mounting the first and second sensors to the patient closely adjacent each other and with a common orientation such that both sensors sense the same noise component.

14. The method according to claim 13, wherein sensing the physiological status includes:
sensing changes in pressure.

15. The method according to claim 12, wherein the sensed physiological status varies in a predefined frequency range and further including:
filtering the first and second signals to remove frequency components above the predefined frequency range.

16. A method for operating an MRI system comprising:
generating static magnetic fields in a patient;
imposing gradient magnetic fields on the static magnetic fields;
imposing radio frequency fields to induce magnetic resonance in the patient;
acquiring magnetic resonance information from the patient and
sensing a physiological status of the patient with the method according to claim 12.

17. The MRI method according to claim 16, further including:
using the physiological status component to control imposing the gradient magnetic fields and the radio frequency fields to acquire the magnetic resonance information only when the patient has a selected physiological status.

18. The MRI method according to claim 16, further including:
reconstructing the acquired magnetic resonance information into images; and
using the physiological status component to control the reconstructing to reconstruct the images from magnetic resonance information when the patient had one or more selected physiological status.

19. A pressure sensing device for sensing a pressure signal and minimizing an amount of interference generated during a magnetic resonance (MR) scan in a magnetic resonance (MR) system having a bore, comprising:
a first active piezoresistive sensor located proximate to the bore of the MR system that is configured to sense a pressure stimulus for the pressure signal to be generated thereat and the amount of interference generated, comprising a first differential output;
a second non-active sensor adjacent to and electromagnetically coupled to the first sensor that is configured to sense the amount of interference generated only, comprising a second differential output; and
an interference filter unit that is electrically coupled to the first differential output and the second differential output of the first sensor and the second sensor respectively, and configured to subtractively cancel the amount of interference from the first and the second differential output; and
a transmitter device coupled to the interference filter unit and configured to wirelessly transmit the pressure signal.

20. The device of claim 19, wherein the first sensor and the second sensor comprises a pressure transducer respectively located inside the bore and adjacent to one another in a same direction and a same axis so that the first sensor and the second sensor are exposed to the amount of interference comprising a substantially equal amplitude and phase at each sensor, and configured to invasively sense pressure stimuli for an invasive pressure monitoring device, wherein the first sensor comprises a Wheatstone bridge circuit that is configured to output a differential-mode signal that is proportional to a change in pressure.
